(19) United States
(12) Patent Application Publication
(10) Pub. No.: US 2003/0139737 A1
(43) Pub. Date: Jul. 24, 2003

(54) Method and apparatus for treatment of presbyopia by lens relaxation and anterior shift

(76) Inventor: J.T. Lin, Oviedo, FL (US)

Correspondence Address: J.T. Lin, 4532 Old Carriage Trail, Oviedo, FL, 32765, US

(21) Appl. No.: 10/053,593

(22) Filed: Jan. 24, 2002

(51) Int. Cl. A61B 18/18; A61B 19/00

(52) U.S. Cl. 606/5; 606/4; 128/898

(57) ABSTRACT

A surgical method and apparatus for presbyopia correction removal of the sclera tissue are disclosed. Mechanisms based on sub-conjunctiva filled-in of the sclera area and cause the sclera-ciliary-body and zonule “complex” become more flexible (or less rigidity) are proposed. Total accommodation based a lens relaxation and lanes anterior shift is calculated and proposed as the guidance of the parameters for device design and clinical outcomes The preferred embodiments for the ablation patterns include radial lines, curved lines, ring dots or any non-specific shapes in a symmetric geometry. The surgery apparatus includes non-laser device of radio frequency wave, electrode device, bipolar device and plasma assisted device. Another preferred embodiment is to use post-operation medication such as pilocarpine (1%-10%) or medicines with similar nature which may cause ciliary body contraction for more stable and enhancement after the treatment.

### BACKGROUND OF THE INVENTION

1. Field of the Invention

The present invention relates to method and apparatus for the treatment of presbyopia by changing the rigidity property of the sclera-ciliary complex and accommodation caused by lens relaxation and anterior shift.

2. Prior Art

Corneal reshaping including a procedure called photorefractive keratectomy (PRK) and a new procedure called laser assisted in situ keratomileusis, or laser intrastroma keratomileusis (LASIK) have been performed by lasers in the ultraviolet (UV) wavelength of (193-213) nm. The commercial UV refractive lasers include ArF excimer laser (at 193 nm) and other non-excimer, solid-state lasers such as those proposed by the present inventor in 1992 (U.S. Pat. No. 5,144,630) and in 1996 (U.S. Pat. No. 5,520,679). The above-described prior arts using lasers to reshape the corneal surface curvature, however, are limited to the corrections of myopia, hyperopia and astigmatism.

Refractive surgery using a scanning device and lasers in the mid-infrared (mid-IR) wavelength was first proposed by the present inventor in U.S. Pat. No. 5,144,630 and 5,520,679 and later proposed by Telfair et. al., in U.S. Pat. No. 5,782,822, where the generation of mid-IR wavelength of (2.5-3.2) microns were disclosed by various methods including: the Er:YAG laser (at 2.94 microns), the Raman-shifted solid state lasers (at 2.7-3.2 microns) and the optical parametric oscillation (OPO) lasers (at 2.7-3.2 microns).^{a}

Corneal reshaping may also be performed by laser thermal coagulation currently conducted by a Ho:YAG laser (at about 2 microns in wavelength) proposed by Sand in U.S. Pat. No. 5,484,432. This method, however, was limited to low-diopter hyperopic corrections. Strictly speaking this prior art did not correction the true “presbyopia” and only performed the mono-vision for hyperopic patients. A thermal laser is required and the laser treated area was within the optical zone diameters of about 7 mm.

The prior art, however, did not actually resolve the intrinsic problems of presbyopic patient caused by age where the cornea lens loss its accommodation as a result of loss of elasticity due to age.

All the above-described prior arts are using methods to change the cornea surface curvature either by tissue ablation (such as in UV laser) or by thermal shrinkage (such as in Ho:YAG laser) and all are using lasers onto the central potion of the cornea.

The alternative method for presbyopia correction, therefore, is to increase the accommodation of the presbyopic patients by change the intrinsic properties of the sclera and ciliary tissue to increase the lens accommodation without changing the cornea curvature.

To treat presbyopic patients using the concept of expanding the sclera by sclera expansion band (SEB) has been proposed by Schachar in U.S. Pat. Nos. 5,489,299, 5,722,952, 5,465,737 and 5,354,331. These mechanical approaches have the drawbacks of complexity and are time consuming, costly and have potential side effects. To treat presbyopia, the Schachar U.S. Pat. Nos. 5,529,076 and 5,722,952 propose the use of heat or radiation on the corneal epithelium to arrest the growth of the crystalline lens and also propose the use of lasers to ablate portions of the thickness of the sclera However, these prior arts do not present any details or practical methods or laser parameters for the presbyopic corrections. No clinical studies have been practiced to show the effectiveness of the proposed concepts. The concepts proposed in the Schachar U.S. Pat. Nos. 5,354,331 and 5,489,299, regarding lasers suitable for ablating the sclera tissues were incorrect because he did not identify which lasers are “cold lasers”. Many of his proposed lasers are thermal lasers which will cause thermal burning of the cornea, rather than tissue ablation. Furthermore, the clinical issues, such as locations, patterns and depth of the sclera tissue removal were not indicated in these prior patents. In addition, it is essential to use a scanning or fiber-coupled laser to achieve the desired ablation pattern and to control the ablation depth on the sclera tissue. Schachar's methods proposed in his prior arts also require the weakening of the sclera and increasing of lens diameter for patient's accommodation. The new mechanisms proposed in the present invention, on the contrary, propose that lens diameter decreases and anteriorly shifted when accommodation occurs to see near. In addition, no implant is needed in the present invention (based on non-expansion theory), which is required in Schachar's based on expansion theory.

Another prior art proposed by Spencer Thornton (Chapter 4, “Surgery for hyperopia and presbyopia”, edited by Neal Sher (Williams & Wilkins, MD, 1997) is to use a diamond knife to incise radial cuts around the limbus areas. It requires a deep (90%-98%) cut of the sclera tissue in order to obtain accommodation of the lens. This method, however, involves a lot of bleeding and is difficult to control the depth of the cut which requires extensive surgeon's skill. Another drawback for presbyopia correction provided by the above-described incision-method is the major post-operative regression of about (30%-80%). The regression is minimum in the ablation-method proposed in the present invention. We note that there is intrinsic difference between the ablation-method proposed in this invention and the knife-incision-method. The sclera space produced by the incision-method is not permanent (unless implantation like Schachar is used) and this space will be reduced during the tissue healing and cause the regression. This major source of regression in incision-method and in Schchar's SEB method, however will not occur in the ablation-method proposed in this invention, where portion of the sclera tissue is permanently removed and filled by the naturely-grown sub-conjunctiva tissue of the eye, rather than the implanted plastic bands in prior arts.

The prior arts of the present inventor, U.S. Pat. Nos. 6,258,082 and 6,263,879 both required the use of a laser to remove portion of the sclera tissue and both are based on the concept of “lens relaxation”, where the scleral ablation causes the ciliary body to contract for lens relaxation to see near. From the recent clinical results using lasers proposed in the above two prior arts, we found that the concept of lens relaxation as the sole mechanism for accommodation is not sufficient. There are cases where the lens capsule is hard to relax or reshape its curvature, we still expect patients with accommodation to see near. Therefore new mechanisms are required which does not require a laser but only an ablation means and additional accommodation mechanisms. The important concept proposed in the present invention is to support the post-operative results which show: (a) minimum regression and (b) lens relaxation is not always required for accommodation. To explain minimum regression, we proposed that the ablated sclera tissue “gap” is filled in by the sub-conjunctival tissue within few days after the surgery. This filled in sub-conjunctival tissue is much more flexible than the original sclera tissue. Therefore the filled-in gap in the sclera area will cause the sclera-ciliary-body and zonule “complex” become more flexible (or less rigidity) after the treatment. We further propose that this elastic “complex” may cause two mechanisms: (a) lens relaxation (LR) or curvature increase and (b) lens anterior shift (AS). Patient's total accommodation amplitude (AA) is therefore attribute to both LR and AS. In addition, the AA may also attribute to the increase of globe axial length after the treatment.

One objective of the present invention is to provide an apparatus and method to obviate the drawbacks in the prior arts.

It is yet another objective of the present invention to provide new mechanisms which support minimum regression and lens anterior shift after sclera ablation by a non-laser method.

It is yet another objective of the present invention to provide a theoretical modeling and calculation to predict the accommodation amplitude (AA) versus the lens radii changes and anterior chamber depth and a “dynamic” model which includes both lens relaxation and lens shift.

It is yet another objective of the present invention to provide parameters for ablation pattems, depth and width required for sufficient accommodation.

It is yet another objective of the present invention to provide a medication method for stable and/or enhanced results after the treatment.

The concept presented in the present patent is to remove portion of the sclera tissue which is filled in by sub-conjunctiva tissue to increase the flexibility of the scleral area and in turn causes the zonular fiber to increase the lens accommodation. Therefore for sufficient accommodation, one to remove enough volume of scleral tissue defined by the ablation width times depth times depth and this “gap” may be filled in by the sub-conjunctival tissue. These non-laser methods shall include, but not limited to, physical blades or knife, electromagnetic wave such as radio frequency wave, electrode device, bipolar device and plasma assisted electro-surgical device.

The invention having now been fully described, it should be understood that it may be embodied in other specific forms or variations without departing from the spirit or essential characteristics of the present invention. Accordingly, the embodiments described herein are to be considered to be illustrative and not restrictive.

### SUMMARY OF THE INVENTION

The preferred embodiments of the basic ablation means of the present invention shall include: physical blades or knife, electromagnetic wave such as radio frequency wave, electrode device, bipolar device and plasma assisted electro-surgical device.

It is yet another preferred embodiment to open the conjunctiva layer (the “flap”) prior to the ablation of the under-layer sclera tissue for a better control of the ablation depth and for safety reasons. This flap is replaced to cover the ablated sclera area.

It is yet another preferred embodiment is that the conjunctiva layer may be lifted to generate the “gap” for the ablation device to insert into the gap and ablate the desired patterns underneath and to avoid or minimize bleeding or infection.

It is yet another preferred embodiment is that both conjunctiva layer and sclera tissue are ablated by the ablation means without the need of conjunctiva flap.

It is yet another preferred embodiment is that the ablation patterns on the sclera area include radial lines, curved lines, ring-dots or any non-specific shapes in a symmetric geometry.

It is yet another preferred embodiment is to use post-operation medication such as pilocarpine or medicines with similar nature which may cause ciliary body contraction to stable and/or enhance the post-operative results after the ablation-method.

Further preferred embodiments of the present invention will become apparent from the description of the invention which follows.

### BRIEF DESCRIPTION OF THE DRAWINGS

FIG. 1 shows the filling effect of the sub-conjunctival layer over the removed sclera area.

FIG. 2 shows the ablation area outside the limbus.

FIG. 3 shows various the ablation patterns on the scleral tissue outside limbus.

FIG. 4 shows the accommodation due to lens relaxation and anterior shift.

### DETAILED DESCRIPTION OF THE INVENTION AND THE PREFERRED EMBODIMENTS

A surgical system in accordance with the present invention comprises a basic tissue removal or ablation means includes physical blades or knife, electromagnetic wave such as radio frequency (RF) wave, electrode device, bipolar device and plasma assisted electro-surgical device. When the above radio frequency devices are used, the preferred embodiment requires a minimum thermal damage to the sclera tissue with efficient ablation which can be controlled by its frequency (10 to 1000) KHz, pulse duration, 100 micro-seconds to continuous wave, and its power output (0.1-20) W. The “ablation” is defined in a general sense to include tissue removal by means of incision or evaporation. The dimension of the removed sclera tissue, its depth, width and length may be easily controlled by the size of the physical blades or the end tips of the ratio frequency (RF) device. The RF wave may be generated by a sinusoidal, square or no-specified shapes.

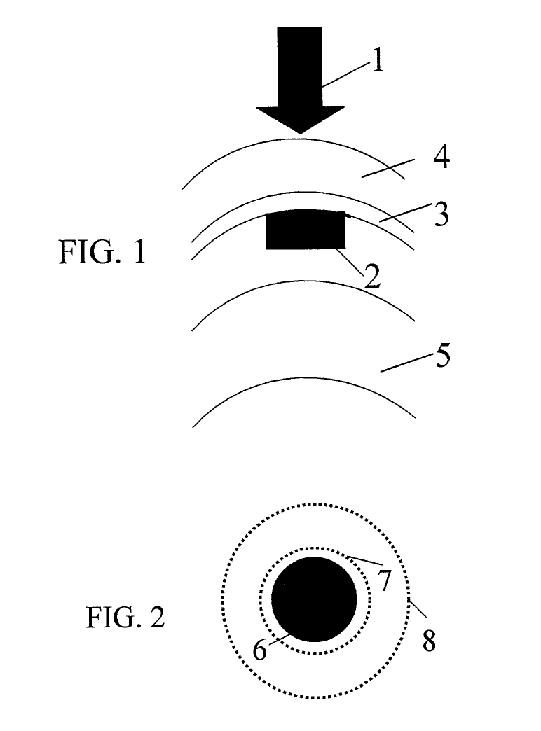

FIG. 1 shows the filling effect of the sub-conjunctival layer over the removed sclera area. The tissue removal means 1 is used to remove a portion of the sclera 2 which is then filled by the sub-conjunctival tissue 3. The first layer is conjunctiva 4 and the layer underneath the sclera 2 is ciliary body 5

FIG. 2 shows the ablation area outside the limubus 6 and within two circles 7,8 having a diameter of 9.0 and 20 mm, respectively.

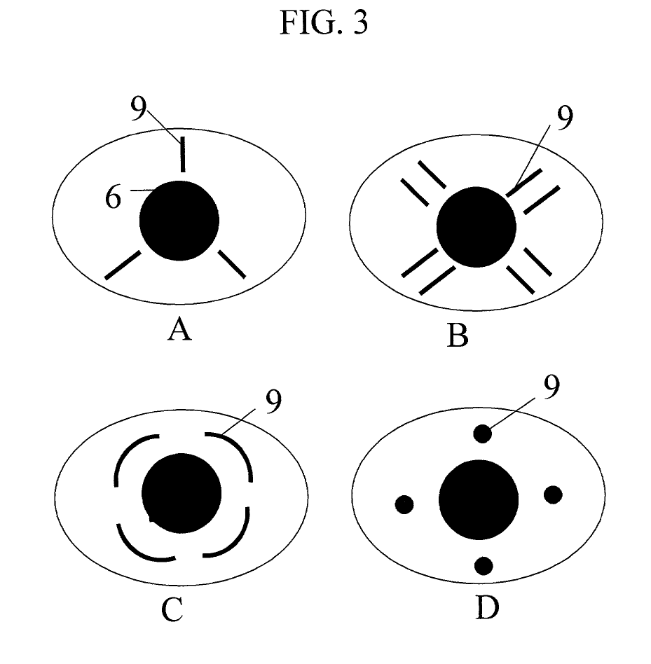

FIG. 3 shows various the ablation patterns 9 generated on the scleral area about (0.1-1.0) mm posterior to the limbus 6. The preferred patterns of this invention include a ring-spot having at least one ring with at least 3 spots in each ring having spot size of (02.-4.0) mm in diameter, and a radial-line or curved line around the limbus having at least 3 lines and each line having a depth of (200-800) microns, a width of (0.2-3.0) mm and a length of (1.0-6.0) mm. The preferred area of ablation is defined by an area within two circles having diameters about 9 mm and 20 mm and outside the limbus.

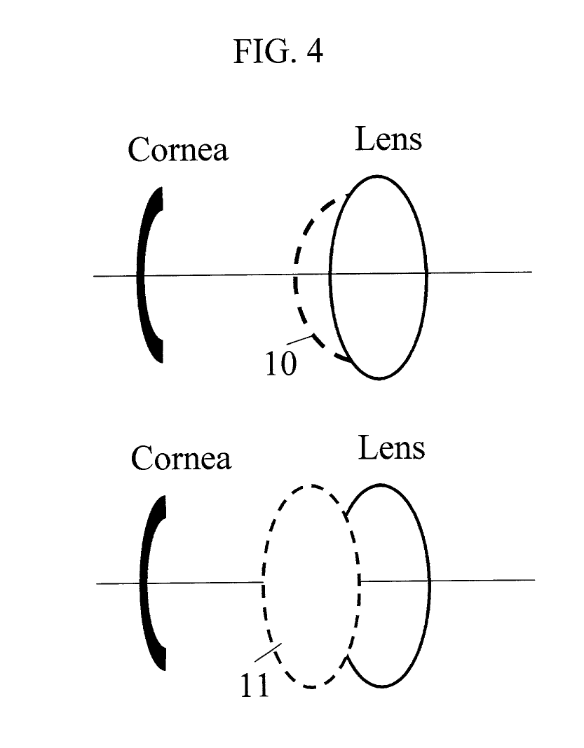

FIG. 4 shows the accommodation due to lens relaxation (LR) with lens curvature 10 change and anterior shifted (AS) 11. Our calculations showed that a typical accommodation post-operation of 2.0 D may be achieved by either (a) lens relaxation (LR) with decreased radius R1 from 10.56 to 9.0 mm without anterior shift (AS); or (b) combining LR (with R1 decreased to 9.5 mm, total power increase of 0.9 D) and AS of 1.0 mm (with a total power increase of 1.1 D). Greater details of the theoretical background and our modeling are shown as follows.

The effective focal length (F) of an eye which defines the image position at a given set of condition of the cornea, lens and the refractive indices of each portion of the eye: no (for cornea), n1 (for anterior chamber), n (for the lens) and n2 (for the vitreous chamber). The curvature radii of the cornea (and lens) are given by r1 (and R1) for the anterior surface, and r2 (and R2) for the posterior surface. By the geometry theory of image (Born M, Wolf E, Principles of Optics, New York, Macmillan, 1964), we obtain the following equation:

n2/F=n1/f1+n2/f2−n2S/(f1 f2). (1)

The total refractive power D (for humor index n1=n2=1.336) may be expressed by

D=1336/L=1336/[F+L2]. (2)

Where L is the image position (from cornea surface), F is the effective focal length and L2 is the principal plan position (from the cornea anterior surface). We may further express D in terms of lens power (D_{L}) and cornea power (D_{C}) as

D=D_{C}+[1−S/f1]D_{L} (3)

Therefore the total refractive power change (dD) is given by (J. T. Lin, unpublished data, 2002):

dD=dD_{C}[1−S/f2]+dD_{L}[1−S/f1]−1336 dS/(f1 f2)−dP+dN, (4)

where dS=S′−S is the effective anterior chamber depth change; dN is the power change cause by refractive index change; dP is the power reduction factor due to the principal plan shift caused by lens curvature, thickness changes and lens shift.

Given an initial axial length of L=24, we may easily derive the power change due to dL (assuming all other parameters remain) as follows:

dD=−1336 dL/L²=−2.32 dL. (5)

Above equation provides us a diopter increase of 2.32 D per mm decrease of the globe axial length. We may propose that accommodation may be achieved by a procedure which may increase the axial length to see near dynamically. Accommodation of 2.0 D corresponding to an increase of axial length of 0.86 mm

The total power changes due to dS and lens curvature changes are more complicate and require computer calculation because the change from the principal plan shift, dP in Eq. (4).

Our numerical results show that (a) For a given R1=10.56, each 1.0 mm anterior lens shift dS, the total power increased by dD=(0.97, 1.04, 1.10) depending on the cornea focal length f1=(34, 31, 28); (b) dD also depends on the values lens radii. For R1=(10.56 to 4.5), dD=(1.1 to 2.90) for f1=28; dD=(1.04 to 1.97) for f1=31; and dD=(0.97 to 1.87) for f1=34. That is a patient with smaller lens power or cornea power (or longer focal length f1 or f2) will have more power change for a given distance of lens shift. For a typical patient lens, a power increase of about 1.4 diopters for R1=10.0 mm versus 1.8 diopters for R1=9.0 mm, for 1.0 mm lens anterior shift (dS=−1.0 mm).

For total accommodation amplitude (AA), we propose the “Lin dynamic model” by introducing two components, the anterior shift (AS) and lens relaxation (LR) to count for the AA. Our calculations showed that one mm AS of the lens will cause about (0.97 to 2.0) diopter of image myopic shift for patients to see near and the reversed process, posterior shift (PS) will allow the patient to see far. We note that these AS and PS are “dynamical” effects allowing the lens to move forward and backward for a presbyopic patient to accommodate both near and distance vision. The second component LR causes the presbyopic lens to see near by lens relaxation with decreased radii of the lens, mainly by the anterior capsule of the lens. For a typical post-surgery patients with an average accommodation amplitude (AA) of +2.0 D, we propose that these results may attribute to AS or LR or the combination of them.

Our numerical calculation showed an increase of AA=2.0 D maybe achieved by any of the following: (a) lens relaxation (LR) with decreased radius R1 from 10.56 to 9.0 mm without AS; (b) combining LR with R1 decreased to 9.5 mm (with a lens power change of 1.36 D and total power increase of 0.9 D) and AS of 1.0 mm (with a total power increase of 1.1 D); and (c) an anterior shift (AS) of about 1.9 mm without LR.

Although the lens power change is very sensitive to its radii changes, about (0.8 to 2.7) D/mm as shown by our calculations, its effect on AA however is limited by factors of the rigidity of the lens capsule (RLC) and the available amount of ciliary contraction and its spacing to the lens, and the zonular effective length (ZEL) defined by the amount of expansion to allow lens central curvatures (radii) change. The AA given by anterior lens shift (AS) on the other hand is not limited or affected by the condition of RLC, therefore it is still possible to have sufficient AA (say +1.0 to 1.5D) by a pure AS without the help from LR, particularly for lens with initial radii smaller than 9.5 by noting that the AA is inverse proportional to the initial value of (R1, R2).

Clinically, it is import to note that the total accommodation amplitude (AA) is governed by the amount of ciliary body contraction, therefore the AA shall be governed by the amount (or volume) of sclera tissue removed, rather than just the depth or length of the ablation. And a minimum threshold (TH) of sclera tissue must be removed in order to have efficient M for the patient to see near at about 35 cm. From the empirical data of Glasser A and Campbell MCW (Presbyopia and the optical changes in the human crystalline lens with age. Vis Res Vol 38:209-229, 1998) and our calculated data, we find that the change of accommodation amplitude (AA) versus ciliary body contraction distance (C) is non-linear in nature The AA per mm change of C, or Mc, depends on the value of C as follows: Mc=(1.94, 2.3, 2.6, 1.0, 0.8) D/mm, for C ranges of C=(0.0-0.5), (0.6-1.0), (1.1-1.5), (1.5-2.0) and (2.0-2.5) mm Based on these data, we further propose that a minimum volume of sclera tissue about (10-15) mm cubic will be required for M to be about 2.0 D.

We note that without the above theoretical calculations and modeling, it would be very difficult to predict the accommodation amplitude and the new mechanisms based on LR and AS. Our method in this invention and parameters for the proposed device and clinical techniques are based upon the above theoretical findings.

The ablation depth of the sclera ciliary tissue is about (200-800) microns and adjustable according to the optimal clinical outcomes including minimum regression and maximum accommodation for the presbyopic patients. The preferred radial ablation shall start at a distance about (4.0-5.5) mm from the corneal center and extended about (2 0-5.0) mm outside the limbus. The preferred embodiments of the radial patterns on the sclera area include at least 3 radial lines, curved lines, ring-dots or any non-specific shapes in a symmetric geometry as shown in FIG. 3.

One preferred embodiment is to coagulate the conjunctiva layer and then prepare a “flap” by cutting (by a blades or other means) a half-circle over the conjunctiva surrounding the limbus with a diameter about 10 mm which is then pushed aside in order for the ablating device to cut the sclera layer underneath. It is also possible to use the ablating device to cut both conjunctiva layer and sclera tissue.

Another preferred embodiment is not to open the conjunctiva layer, but to insert the fiber tip through the conjunctiva layer and ablate the sclera tissue underneath such that the procedure is done non-invasively. To do this procedure, the conjunctiva layer may be lifted to generate the “gap” for fiber tip to insert into the gap and ablate the desired patterns underneath. Additional advantages of this invasive method is to avoid or minimize bleeding or infection. We note that most of the bleeding is due to cutting of the conjunctiva tissue rather than the ablation of the sclera tissue.

The preferred embodiment for these non-laser methods shall include, but not limited to, physical blades or knife, electromagnetic wave such as radio frequency wave, electrode device, bipolar device and plasma assisted electrode device. The electromagnetic wave generator is commercially available However, the parameters of the device such as its frequency, pulse duration and repetition rate and the size of the electrode tip shall be selected for efficient cutting (or ablation) with minimum thermal damage to the tissue to be removed.

Another preferred embodiment is to use post-operation medication such as pilocarpine (1%-10%) or medicines with similar nature which may cause ciliary body contraction. These post-operation medicine will cause more stable, less regression and/or enhancement after the treatment. The total accommodation short after the procedure using the medicine shall include the tissue removal effects and the effect due to medicine (contraction). Long terms results shall be mainly due to tissue removal with enhanced initially by the medicine. The initial ciliary contraction enhancement is important for stable long terms results to prevent regression caused by tissue healing, before the permanent sub-conjunctiva filling completion.

While the invention has been shown and described with reference to the preferred embodiments thereof, it will be understood by those skilled in the art that the foregoing and other changes and variations in form and detail may be made therein without departing from the spirit, scope and teaching of the invention. Accordingly, threshold and apparatus, the ophthalmic applications herein disclosed are to be considered merely as illustrative and the invention is to be limited only as set forth in the claims.

I claim:

1. A surgical method for treating presbyopic patient by removing a portion of the sclera tissue of an eye comprising the steps of:
selecting a tissue removal means; and
controlling said tissue removal means to remove sclera tissue in a predetermined pattern in predetermined area of an eye outside the limbus, whereby patient's accommodation amplitude increases to improve near vision.

2. A surgical method as claimed in claim 1, wherein said accommodation amplitude increase is caused by the change of the elastic property of the sclera-ciliary-zonules complex.

3. A surgical method as claimed in claim 1, wherein said accommodation amplitude increase is caused by the contraction of said complex.

4. A surgical method as claimed in claim 1, wherein said accommodation amplitude increase is caused by lens relaxation or changing of lens curvatures.

5. A surgical method as claimed in claim 1, wherein said accommodation amplitude increase is caused by lens anterior shift.

6. A surgical method as claimed in claim 1, wherein said accommodation amplitude increase is caused by the increase of globe axial length.

7. A surgical method as claimed in claim 1, wherein said accommodation amplitude increase is further caused by post-operation medicine includes pilocarpine or medicines in similar nature causing the ciliary body or said complex contraction.

8. A surgical method as claimed in claim 1, wherein said tissue removal means includes a physical blades.

9. A surgical method as claimed in claim 1, wherein said tissue removal means includes an electromagnetic wave at radio frequency ranging of (10-1000) KHz and power of (0.1-20) W.

10. A surgical method as claimed in claim 1, wherein said tissue removal means includes an electrode device, or a bipolar device, or plasma assisted electrode device.

11. A surgical method as claimed in claim 1, wherein said predetermined pattern includes radial lines, curved lines, ring dots or any non-specific shapes in a symmetric geometry.

12. A surgical method as claimed in claim 1, wherein said predetermined area includes area outside the limbus defined by the area between two circles having diameters of 9 mm and 20 mm.

13. A method as claimed in 11, wherein said radial lines include at least 3 lines each line having a depth of (200-800) microns, a width of (0.2-3.0) mm and a length of (1 0-6.0) mm.

14. A method as claimed in 11, wherein said curved lines include at least 3 curved lines each line having a depth of (200-800) microns, a width of (0.2-3.0) mm and a length of (1.0-6.0) mm.

15. A method as claimed in 11, wherein said ring dots include at least 3 ring dots each dot having a depth of (200-800) microns and a diameter of (0.2-4.0) mm.
